(19) United States
(12) Patent Application Publication
(10) Pub. No.: US 2011/0060389 A1
(43) Pub. Date: Mar. 10, 2011

(54) METHOD FOR CORRECTION OF FEMALE INCONTINENCE AND RECONSTRUCTION OF TISSUE

(76) Inventor: Julie Reil, Billings, MT (US)

(21) Appl. No.: 12/753,600

(22) Filed: Apr. 2, 2010

Related U.S. Application Data
(60) Provisional application No. 61/165,993, filed on Apr. 2, 2009.

(51) Int. Cl. A61F 7/08

(52) U.S. Cl. 607/96

(57) ABSTRACT

The method of the present invention is used for treating symptoms of Stress Urinary Incontinence by the collective reduction of six anatomical areas of the female external genitalia. The following six treatment areas are included in the procedure: the labia majora area, the labia minora area, the clitoral hood area, the periurethral area, the vaginal introitus area, and the perineal area. For each of the six anatomical areas treated in the method there is a standard procedure that is unique to that area.

### RELATED APPLICATIONS

This application claims priority from Provisional Application 61/165,993 filed Apr. 2, 2009.

### BACKGROUND OF THE INVENTION

1. Field of the Invention

The present invention relates to a method of correcting female stress urinary incontinence (SUI) by treating anatomical areas of female external genitalia.

2. Description of the Related Art

Urinary incontinence affects millions of women worldwide. Stress induced urinary incontinence (SUI) has a variety of causes—aging, vaginal deliveries, hormone level changes, etc. and can occur as a result of a variety of activities, such as athletic activities, coughing, sneezing, etc. SUI occurs when there is insufficient support of bladder and vaginal tissue.

It is well known to treat SUI with surgery, medication or collagen injections. However, all of these treatments have side effects and/or inherent risks. Therefore, it is desirable to provide a tightening treatment for SUI and a variety of other external genitalia issues.

### SUMMARY OF THE INVENTION

The method of the present invention can be used to treat symptoms of SUI and to to improve the cosmetic appearance of the female external genitalia by reducing skin laxity. For example, the method of the present invention encompasses labia majora reduction, labia minora reduction, clitoral hood reduction, periurethral reduction, vaginal introitus reduction and perineal reduction.

The method of the present invention is used for treating symptoms of Stress Urinary Incontinence by the collective reduction of six anatomical areas of the female external genitalia. The following six treatment areas are included in the procedure: the labia majora area, the labia minora area, the clitoral hood area, the periurethral area, the vaginal introitus area, and the perineal area. For each of the six anatomical areas treated in the method there is a standard procedure that is unique to that area.

As will be described fully herein the method of the present invention can also be used for cosmetic improvement of the female external genitalia. One to all six anatomical areas can be treated based upon a provider's clinical assessment of which anatomical areas would benefit from treatment.

The method can be modified slightly to treat labia minora asymmetry. The method can effectively treat discrepancy in size of the labia minora thereby restoring symmetry in the size of the labia minora. The protocol of the present method can be modified to reduce the size of one side of the labia minora compared to the opposite side for treatment of asymmetry.

The method of the present invention is modified slightly to treat labia majora asymmetry. The method can treat discrepancy in size of the labia majora restoring symmetry in the size of the labia majora. The present method can be modified to reduce the size of one side of the labia majora compared to the opposite side for treatment of asymmetry.

The method of the present invention can also be used to treat perineal scars. Using the method of perineal reduction, a perineal scar can be treated for improved cosmetic appearance and treatment of scar asymmetry.

According to these and other aspects of the present invention there is provided a method for the correction of incontinence and reconstruction of tissue, comprising the steps of providing a heating device for applying treatment to designated areas of tissue, reducing the tissue of the labia majora area, reducing the tissue of the labia minora area, reducing the tissue of the clitoral hood area, reducing the tissue of the periurethal area; reducing the tissue of the vaginal introitus area, and reducing the tissue of the perineal area.

These and other features, aspects, and advantages of the present invention will become more apparent from the following detailed description of the preferred embodiment relative to the accompanied drawings, in which:

### BRIEF DESCRIPTION OF THE DRAWINGS

FIG. 1 is a flow chart 10 for implementing the method of the present invention.

FIG. 2 is a flow chart 30 implementing the method for labia majora reduction.

FIG. 3 is a flow chart 50 implementing the method for labia minora reduction.

FIG. 4 is a flow chart 70 implementing the method for clitoral hood reduction.

FIG. 5 is a flow chart 90 implementing the method for periurethal reduction.

FIG. 6 is a flow chart 110 implementing the method for vaginal introitus reduction.

FIG. 7 is a flow chart 130 implementing the method for perineal reduction.

### DETAILED DESCRIPTION OF THE PREFERRED EMBODIMENT

The method of the present invention encompasses the following six treatments: labia majora reduction; labia minora reduction; clitoral hood reduction; periurethral reduction; vaginal introitus reduction and perineal reduction.

For the treatment of Stress Urinary Incontinence (SUI), the method of the present invention includes all six of the above treatment areas collectively. The treatments performed on the six areas listed above are usually performed in the order listed, but are still effective if performed in no particular order. It is the collective reduction of the six areas of the female external genitalia that results in treatment of the symptoms of SUI.

The number of treatments needed to treat symptoms of SUI to the patient's satisfaction will vary according to the severity of the symptoms, age of the patient and the degree of laxity. The treatments typically take approximately 45 minutes to perform. Repeat treatments may be performed as soon as the next calendar day, but preferably two weeks apart.

Some patient's treatment response to method of the present invention, i.e., the GenityTe™ method, is dramatic and can result in rapid improvement of SUI symptoms. Younger patients with younger collagen and elastin tend to achieve faster reduction of treatment areas with and more rapid improvement of SUI symptoms. Older patients, who often have a higher severity of SUI symptoms, older collagen and elastin and more laxity tend to have slower reduction of treatment areas and achieve slower but on gong improvement of SUI symptoms with treatments in series.

In patients who undergo multiple treatments in series for ongoing treatment of their SUI symptoms, the standard treatment protocol should be modified over time according to the anatomic assessment of the provider. The following areas are excluded from the treatment protocol once optimal visible reduction is achieved: the labia majora area, the labia minora area, the clitoral hood area and the perineal area. As optimal visible reduction is achieved of these four areas, the method can be modified to include only treatment using the standard protocol for periurethral area reduction and using a modified protocol for vaginal introitus area reduction which uses five passes on the superior aspect of the vaginal introitus which provides inferior tissue support for the urethra, and 0-5 passes on the lateral and inferior vaginal introitus as needed for laxity treatment. The modifications made when customizing the procedure to exclude certain treatment areas after optimal visible tightening is achieved are referred to as GenityTe™ Advanced Techniques.

The method of the present invention utilizes a broadband light based device (1100-1800 nm) that performs deep dermal heating, for example, the TITAN® by Cutera, Inc. The use of other known deep dermal heating devices is contemplated by the method of the present invention. It should also be appreciated that the present invention contemplates modifications to known light based devices in order to optimize the method of the present invention.

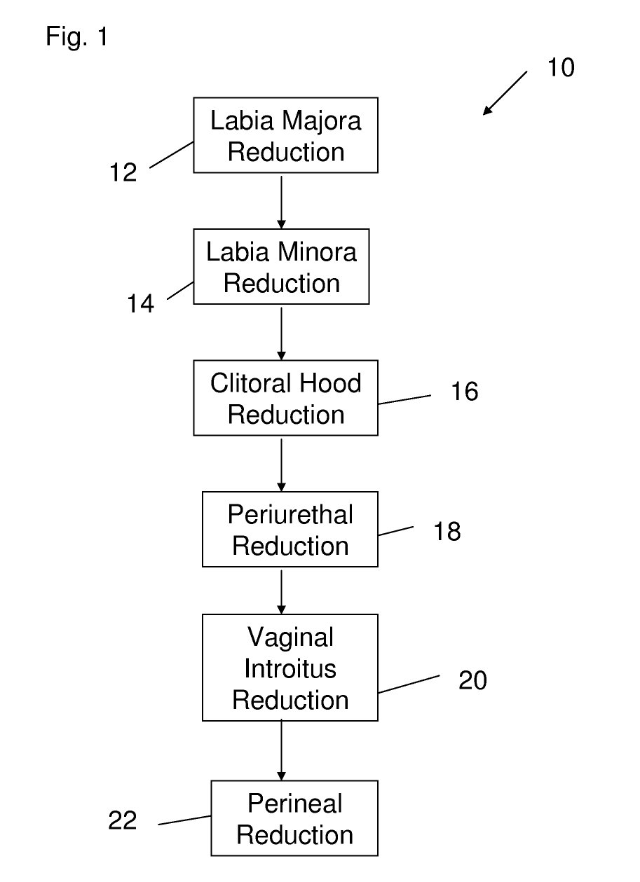

FIG. 1 is a flow chart 10 for implementing the method of the present invention. In the following order the method is performed in the following order at block 12 labia majora reduction occurs. At block 14—labia minora reduction. Clitoral hood reduction is performed next as shown by block 16. At block 18—periurethral reduction. In blocks 20 and 22, vaginal introitus reduction and perineal reduction, respectively. Each of the separate procedures will be described in detail below separately. It should be appreciated that the mechanical operation of the light based device is known and will not be discussed in detail.

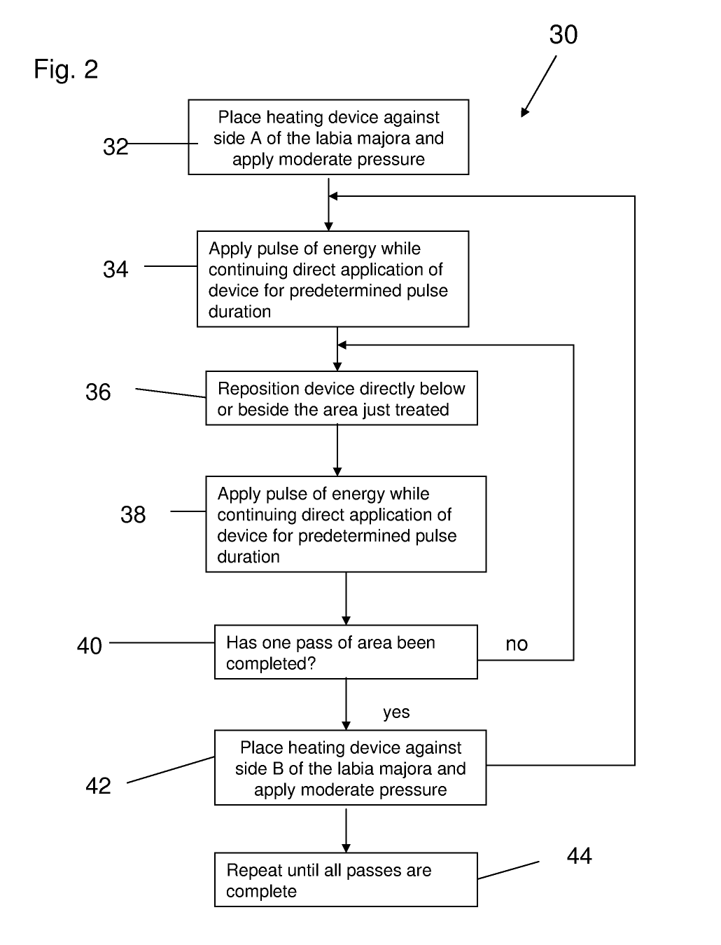

At block 12 reductions of the labia majora is performed by placing a patient in the dorsal lithotomy position on an examining table with stirrups designed for gynecologic examinations. Next, using current medical guidelines from the American College of Obstetricians and Gynecologists for positioning a female patient for a gynecologic exam, the patient is positioned for optimal visualization of the treatment area, guiding the knees apart and the pelvis superiorly as needed. Begin with either the left labia majora or the right labia majora. The side chosen to be treated first is designated as side A. With gloved hands a generous amount (approx. ¼ inch thick) of cool ultrasound gel that has been stored in the refrigerator is applied to the side of the labia majora being treated, side A. Referring to block diagram 30 of FIG. 2, at block 32, using the setting of 32 Joules/cm2, place the heating device (not shown), for example, a sapphire crystal, directly against the skin of the labia majora while applying moderate pressure. The heating device can be positioned either horizontally or vertically, though this orientation should be continued throughout the treatment of the labia majora. At block 34, the foot pedal of the device (not shown) is depressed to begin the pulse of energy while continuing direct application of the device to the skin for the duration of the pulse (approximately seven seconds). The total amount of time is adjustable and encompasses a range of time, thus the present invention should not be limited to a specific time amount.

Next, as shown in block 36, the device is re-placed directly below or beside the area just treated with minimal overlap of the previously directed pulse. Again, in block 38, the foot pedal is depressed to begin the approximately seven second pulse of energy (see above) while continuing skin contact with moderate pressure applied throughout the duration of the pulse. The skin of the entire treatment area is treated with moderate overlap of the preceding pulse until the entire treatment area has been treated. Steps 36 and 38 are repeated until the entire area has been covered with a pulse, this is referred to as a completed ‘pass’. At block 40 when one pass of the treatment area, side A, is completed, “yes,” the device is used to treat side B. At this time, the used gel is wiped away and disposed, for example, the used gel can be wiped onto a disposable medical blue pad.

In preparation for treatment indicated by block 42, approximately ¼ inch of cool gel is applied to the opposite side, side B, of the treatment area as described above and using the exact protocol outlined above another series of pulses with minimal overlapping of the individual pulses to complete the first pass on the opposite side of the labia majora occurs. When the first pass is complete on the opposite side, side B, of the treatment area the used gel is wiped off and disposed of. For example, as shown in block 44, additional passes of both side A and side B, for example, four, can be done exactly as described above, for a total of five passes over the both sides of the Labia majora in this treatment area. It should be appreciated that the present invention contemplates a range in the number of passes the provider may take on the treatment area and hence should not be limited by a specific number of passes.

If discrepancy in size of the two labia majora is observed by the provider, the asymmetry can be treated by reducing the total passes performed on the smaller labia majora to range from, for example, 2 to 3 passes depending on the extent of the size discrepancy. Next, the larger labia majora can be fully treated with standard protocol of, for example, five total passes.

Referring again to FIG. 1, at block 14 reduction of the labia minora is implemented. As with the labia majora reduction described above, the procedure begins by placing a patient in the dorsal lithotomy position on an examining table with stirrups designed for gynecologic examinations. Next, using current medical guidelines from the American College of Obstetricians and Gynecologists for positioning a female patient for a gynecologic exam, the patient is positioned for optimal visualization of the treatment area, guiding the knees apart and the pelvis superiorly as needed.

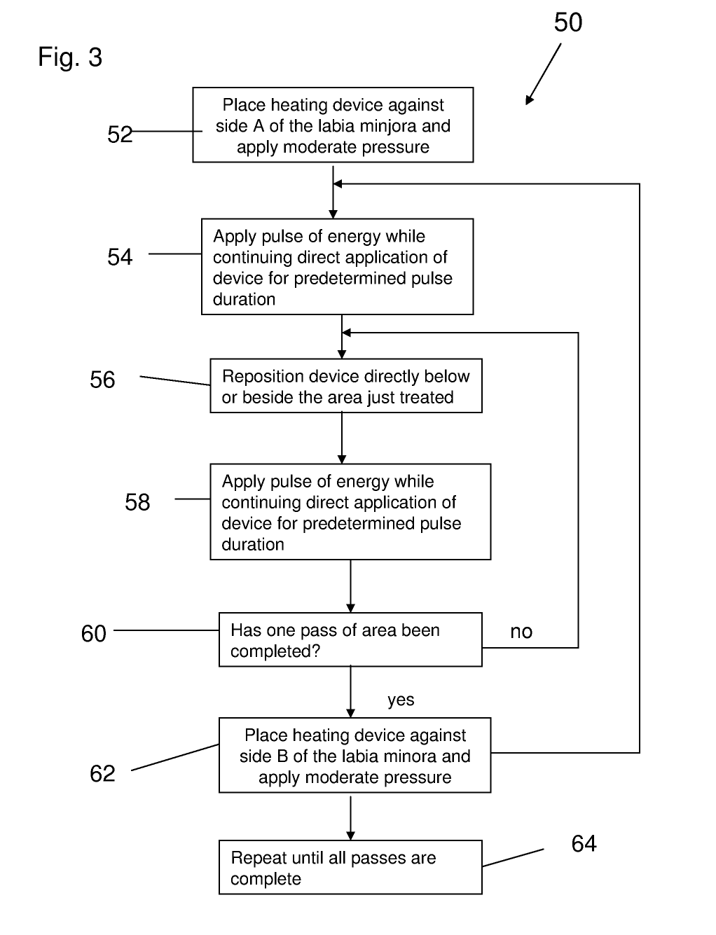

Begin with either the left labia minora or the right labia minora. The side chosen to be treated first is designated as side A. With gloved hands a generous amount (approx. ¼ inch thick) of cool ultrasound gel that has been stored in the refrigerator is applied to the side of the labia majora being treated, side A. Referring to block diagram 50 of FIG. 3, at block 52, using the setting of 32 Joules/cm2, place the heating device (not shown), for example, a sapphire crystal, directly against the skin of the labia minora while applying moderate pressure. The heating device can be positioned either horizontally or vertically, though this orientation should be continued throughout the treatment of the labia minora. At block 54, the foot pedal of the device (not shown) is depressed to begin the pulse of energy while continuing direct application of the device to the skin for the duration of the pulse (approximately seven seconds). The total amount of time is adjustable and encompasses a range of time, thus the present invention should not be limited to a specific time amount.

Next, as shown in block 56, the device is re-placed directly below or beside the area just treated with minimal overlap of the previously directed pulse. Again, in block 58, the foot pedal is depressed to begin the approximately seven second pulse of energy (see above) while continuing skin contact with moderate pressure applied throughout the duration of the pulse. The skin of the entire treatment area is treated with moderate overlap of the preceding pulse until the entire treatment area has been treated. Steps 56 and 58 are repeated until the entire area has been covered with a pulse, this is referred to as a completed “pass.” At block 60 when one pass of the treatment area, side A, is completed, “yes,” the device is used to treat side B. At this time, the used gel is wiped away and disposed, for example, the used gel can be wiped onto a disposable medical blue pad.

In preparation for treatment indicated by block 62, approximately ¼ inch of cool gel is applied to the opposite side, side B, of the treatment area as described above and using the exact protocol outlined above another series of pulses with minimal overlapping of the individual pulses to complete the first pass on the opposite side of the labia minora occurs. When the first pass is complete on the opposite side, side B, of the treatment area the used gel is wiped off and disposed of. For example, as shown in block 64, additional passes of both side A and side B, for example, four, can be done exactly as described above, for a total of five passes over the both sides of the labia minora in this treatment area. It should be appreciated that the present invention contemplates a range in the number of passes the provider may take on the treatment area and hence should not be limited by a specific number of passes.

When discrepancy in size of the two labia minora is observed by the provider, the asymmetry is treated by reducing the total passes performed on the smaller labia minora to range from, for example, 0 to 3 passes depending on the extent of the size discrepancy. Next, the larger labia minora can be fully treated with standard protocol of, for example, five total passes.

Referring again to FIG. 1, in block 16, clitoral hood reduction is the next step in the implementation of the present invention. As in the other treatment protocols, the patient is placed in the dorsal lithotomy position on an examining table with stirrups designed for gynecologic examinations. Next, the patient is positioned for optimal visualization of the treatment area guiding the knees apart and the pelvis superiorly as needed. Beginning superiorly over the clitoral hood, with gloved hands a generous amount (approx. ¼ inch thick) of cool ultrasound gel that has been refrigerated is applied to the area above the clitoris and laterally on either side of the clitoris.

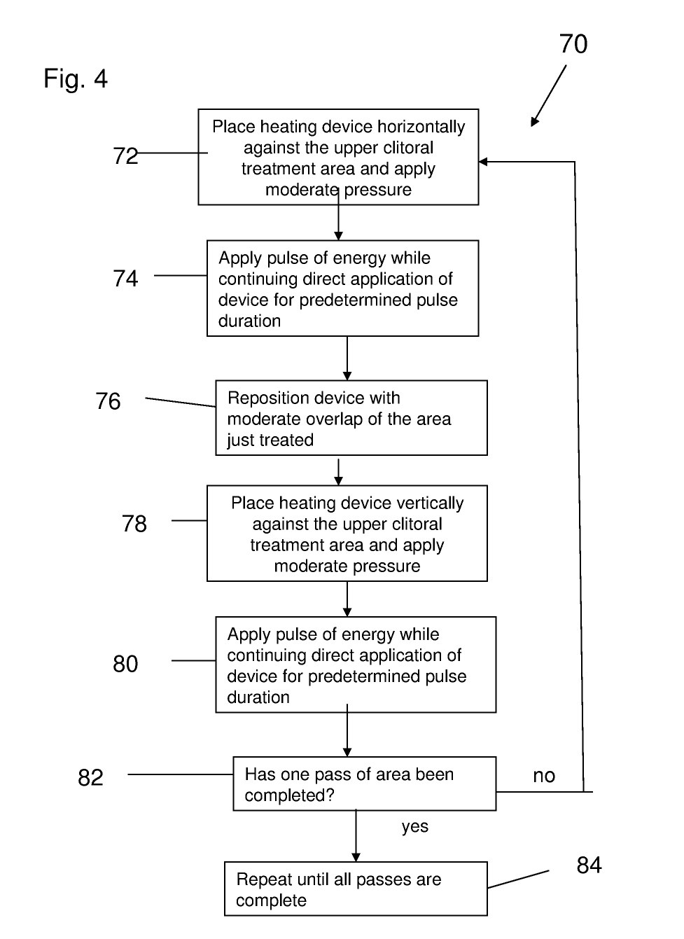

As shown in FIG. 4, block diagram 70 illustrates the implementation of the method steps for clitoral reduction. At block 72, using the setting of 32 Joules/cm2, the device, for example, a sapphire crystal is placed directly against the skin of above the clitoral hood while applying moderate pressure. The device should be orientated horizontally when treating the area above the clitoris. At block 74, the foot pedal of the device (not shown) is depressed to begin the pulse of energy while continuing direct application of the device to the skin for the duration of the pulse (approximately seven seconds). The total amount of time is adjustable and encompasses a range of time, thus the present invention should not be limited to a specific time amount.

In block 76 treatment of the skin of the upper clitoral hood treatment area is continued with moderate overlap of the preceding pulse until the upper clitoral hood treatment area is completed. Next, as illustrated in block 78 and 80 the heating device is orientated vertically and used to treat the area on either side of the clitoral hood as described above. Steps 72-80 are repeated until the entire lateral clitoral hood treatment area and the upper clitoral hood treatment area has been covered with a pulse, this is referred to as a completed “pass.” When one pass of the treatment area is completed as shown in block 82, the used gel can be wiped away gently in a downward direction because of tissue proximity to the sensitive clitoral opening. Approximately ¼ inch of cool gel is reapplied to the treatment area as described above and using the exact protocol outlined above, another series of pulses over the clitoral hood treatment area is implemented as described above.

Block 84 can be repeated, for example, for three more passes exactly as described above, for a total of five passes over this clitoral hood treatment area. It should be appreciated that the present invention contemplates a range in the number of passes the provider may take on the treatment area and hence should not be limited by a specific number of passes.

As shown by block 18 of FIG. 1, the method of the present invention provides a protocol for reducing the periurethral anatomy. The patient is placed in the dorsal lithotomy position on an examining table with stirrups designed for gynecologic examinations and positioned for optimal visualization of the treatment area. Beginning with the skin superior to the urethral opening and inferior to the clitoris a generous amount (approx. ¼ inch thick) of refrigerated ultrasound gel is applied to the area above the urethral opening.

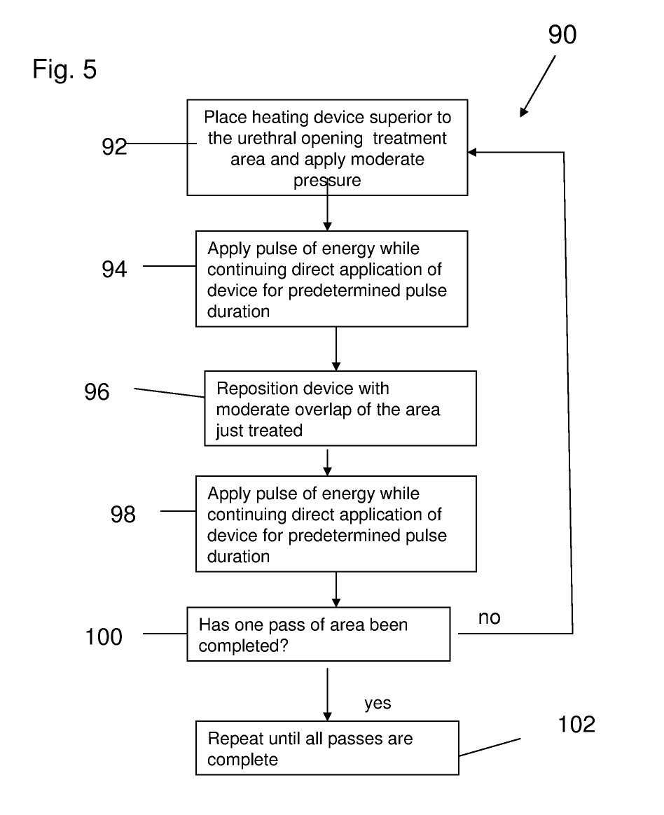

Referring to FIG. 5, in block diagram 90, using the setting of, for example, 32 Joules/cm2, in step 92, the heating device, for example, a sapphire crystal, is placed directly against the skin superior to the urethral opening while applying moderate pressure. The device can be orientated either horizontally or vertically. Typically a horizontal orientation works well beginning superior to the urethra and continuing below the urethra entering the superior vagina to treat the inferior aspect of the urethra for full coverage of the treatment area.

In step 94, a pulse of energy is applied while continuing direct application of the device to the skin for the duration of the pulse (approx. seven seconds). Next, at step 96 the device is repositioned directly below or beside the area just treated with minimal overlap of the previously directed pulse. Again at step 98, the foot pedal is depressed to begin the approx. seven second pulse of energy while continuing skin contact with moderate pressure applied throughout the duration of the pulse.

Continue treating the skin of the entire treatment area with moderate overlap of the preceding pulse until the entire treatment area has been treated. Steps 96 and 98 are repeated until the entire area has been covered with a pulse, this is referred to as a completed “pass,” shown in step 100. When one pass of the periurethral treatment area is completed, the used gel is wiped away and disposed of. Gel can be reapplied gel to the treatment area as described above and using the exact protocol outlined above, begin another series of pulses with minimal overlapping of the individual pulses to complete the second pass of the periurethral area as shown by block 100. As indicated in step 102 the above can be repeated as desired, for example, three more passes can be made exactly as described above, for a total of five passes over the periurethral treatment area.

As shown in FIG. 1, the next recommended treatment step according to the method of the invention is vaginal introitus reduction indicated by block 20. The patient is placed in the dorsal lithotomy position on an examining table with stirrups designed for gynecologic examinations and positioned for optimal visualization of the treatment area. Beginning at one side of the vaginal introitus and continuing circumferentially for 360 degrees. a generous amount (approx. ¼ inch thick) of refrigerated ultrasound gel is applied to the vaginal introitus area.

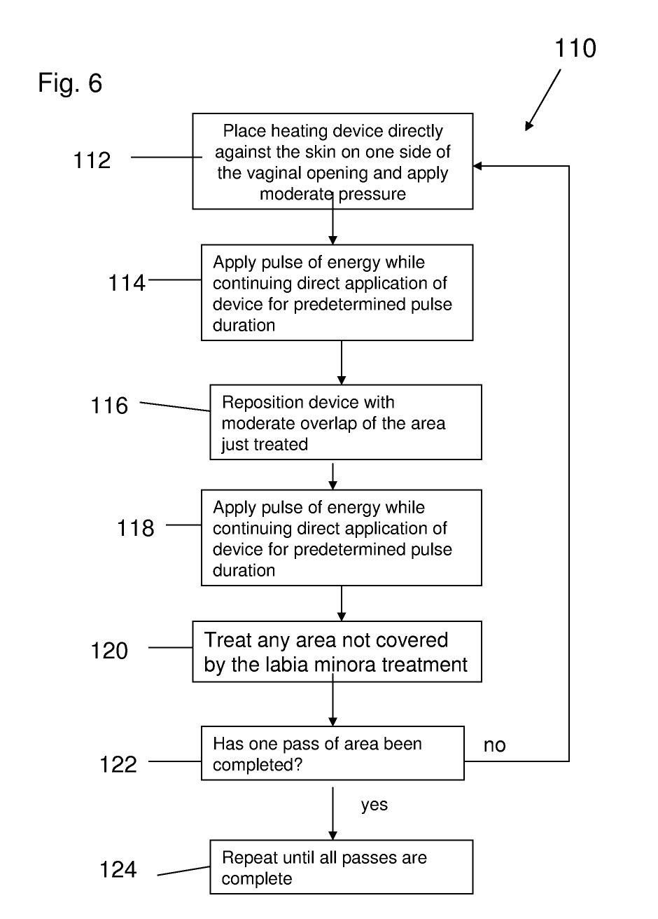

As shown in FIG. 6, in block diagram 110, using a setting of for example, 32 Joules/cm2, the heating device is placed directly against the skin on one side of the vaginal opening at the hymenal ring and moderate pressure is applied at step 112. The heating device can be orientated horizontally and directed inside the hymenal opening. This orientation should be continued when treating the inner hymenal area of the vaginal opening.

In step 114, a pulse of energy is applied while continuing direct application of the device to the skin for the duration of the pulse (approx. seven seconds). Next, at step 116 the device is repositioned directly below or beside the area just treated with minimal overlap of the previously directed pulse. Again at step 118, approximately seven second pulse of energy is applied while continuing skin contact with moderate pressure applied throughout the duration of the pulse.

As indicated treatment of the skin of the entire vaginal opening area with moderate overlap is continued by overlap of the preceding pulse until the entire vaginal opening treatment area has been treated. Next treat any skin in the vaginal opening treatment area not covered in the labia minora treatment protocol as shown by block 120. When the entire area has been covered with a pulse, this is referred to as a completed pass shown at 122. When one pass of the vaginal introitus treatment area is completed, the steps are repeated until the entire area has been covered with a pulse, this is referred to as a completed “pass,” shown in step 122. When one pass of the area is completed, the used gel is wiped away and disposed of. Gel can be reapplied gel to the treatment area as described above and using the exact protocol outlined above, begin another series of pulses with minimal overlapping of the individual pulses to complete the second pass of the vaginal introitus area as shown by block 122. As indicated in step 124 the above can be repeated as desired, for example, three more passes can be made exactly as described above, for a total of five passes over the treatment area.

The remaining treatment area of according to the method of the present invention is perineal reduction as indicated by block 22 of FIG. 1. As with the previous treatment areas the patient is placed in the dorsal lithotomy position on an examining table with stirrups designed for gynecologic examinations and positioned for optimal visualization of the treatment area. Beginning with the skin below the vaginal introitus and above the anus, known as the perineum, a generous amount (approx. ¼ inch thick) of refrigerated ultrasound gel is applied to the perineum.

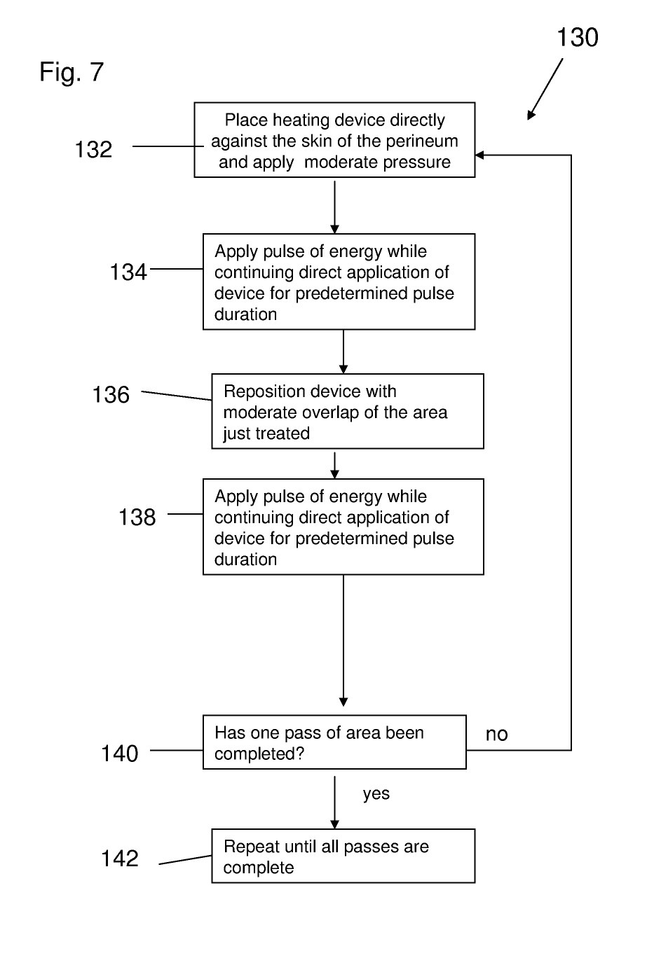

As shown in FIG. 7, in block diagram 130, using a setting of for example, 32 Joules/cm2, the heating device is placed directly against the skin of the perineum while applying moderate pressure in step 132. The device can be orientated either horizontally or vertically throughout the treatment pass. In step 134, a pulse of energy is applied while continuing direct application of the device to the skin for the duration of the pulse (approx. seven seconds). Next, at step 136 the device is repositioned directly below or beside the area just treated with minimal overlap of the previously directed pulse. It is acceptable to include the upper anal area in this treatment area if visible laxity is present.

Again at step 138, approximately seven second pulse of energy is applied while continuing skin contact with moderate pressure applied throughout the duration of the pulse. As indicated treatment of the skin of the area with moderate overlap is continued by overlap of the preceding pulse until the entire treatment area has been treated. When the entire area has been covered with a pulse, this is referred to as a completed pass shown at 140.

When one pass of the treatment area is completed, the steps are repeated until the entire area has been covered with a pulse, this is referred to as a completed “pass,” shown in step 142. When one pass of the area is completed, the used gel is wiped away and disposed of. Gel can be reapplied gel to the treatment area as described above and using the exact protocol outlined above, begin another series of pulses with minimal overlapping of the individual pulses to complete the second pass of the area as shown by block 142. As indicated, the above can be repeated as desired, for example, three more passes can be made exactly as described above, for a total of five passes over the treatment area.

Accordingly, due to the methodology of the present invention SUI and other anatomical issues can be treated by the collective reduction of six anatomical areas of the female external genitalia—the labia majora area, the labia minora area, the clitoral hood area, the periurethral area, the vaginal introitus area, and the perineal area. For each of the six anatomical areas treated in the method there is a standard procedure that is unique to that area.

As set forth above, the method of the present invention can also be used for cosmetic improvement of the female external genitalia. One to all six anatomical areas can be treated based upon a provider's clinical assessment of which anatomical areas would benefit from treatment. The method can also be modified slightly to treat labia minora asymmetry. The method can effectively treat discrepancy in size of the labia minora thereby restoring symmetry in the size of the labia minora. The protocol of the present method can be modified to reduce the size of one side of the labia minora compared to the opposite side for treatment of asymmetry.

The method of the present invention is modified slightly to treat labia majora asymmetry. The method can treat discrepancy in size of the labia majora restoring symmetry in the size of the labia majora. The present method can be modified to reduce the size of one side of the labia majora compared to the opposite side for treatment of asymmetry.

The method of the present invention can also be used to treat perineal scars. Using the method of perineal reduction, a perineal scar can be treated for improved cosmetic appearance and treatment of scar asymmetry.

Although the present invention has been described in relation to particular embodiments thereof, many other variations and modifications and other uses will become apparent to those skilled in the art. It is preferred therefore, that the present invention be limited not by the specific disclosure herein, but only by the appended claims.

What is claimed is:

1. A method for the correction of incontinence and reconstruction of tissue, comprising the steps of:
providing a heating device for applying treatment to designated areas of tissue;
reducing the tissue of the labia majora area;
reducing the tissue of the labia minora area;
reducing the tissue of the clitoral hood area;
reducing the tissue of the periurethal area;
reducing the tissue of the vaginal introitus area; and
reducing the tissue of the perineal area.

2. The method of claim 1, wherein the step of reducing the tissue of the labia majora area comprises placing the heating device against a first and second side of the labia majora and applying a pulse of energy to a treated area.

3. The method of claim 2, wherein the step of reducing the tissue of the labia majora area further comprises repositioning the heating device to moderately overlap the treated area.

4. The method of claim 3, wherein the step of reducing the tissue of the labia majora area further comprises applying a pulse of energy to the repositioned treated area.

5. The method of claim 4, wherein the step of reducing the tissue of the labia majora area further comprises repeating the repositioning and heating steps until each first and second side has been treated.

6. The method of claim 5, wherein the step of reducing the tissue of the labia majora area further comprises completing additional passes of each first and second side with the heating device.

7. The method of claim 1, wherein the step of reducing the tissue of the labia minora area comprises placing the heating device against a first and second side of the labia minora and applying a pulse of energy to a treated area.

8. The method of claim 7, wherein the step of reducing the tissue of the labia minora area further comprises repositioning the heating device to moderately overlap the treated area.

9. The method of claim 8, wherein the step of reducing the tissue of the labia minora area further comprises applying a pulse of energy to the repositioned treated area.

10. The method of claim 9, wherein the step of reducing the tissue of the labia minora area further comprises repeating the repositioning and heating steps until each first and second side has been treated.

11. The method of claim 10, wherein the step of reducing the tissue of the labia minora area further comprises completing additional passes of each first and second side with the heating device.

12. The method of claim 1, wherein the step of reducing the tissue of the clitoral hood area comprises placing the heating device laterally against an upper clitoral treatment area and applying a pulse of energy to a treated area.

13. The method of claim 12, wherein the step of reducing the tissue of the clitoral hood area further comprises repositioning the heating device to moderately overlap the treated area.

14. The method of claim 13, wherein the step of reducing the tissue of the clitoral hood area further comprises applying a pulse of energy to the repositioned treated area.

15. The method of claim 14, wherein the step of reducing the tissue of the clitoral hood area further comprises placing the heating device vertically against an upper clitoral treatment area and applying a pulse of energy to a treated area.

16. The method of claim 15, wherein the step of reducing the tissue of the clitoral hood area further comprises repositioning the heating device to moderately overlap the vertically treated area.

17. The method of claim 16, wherein the step of reducing the tissue of the clitoral hood area further comprises applying a pulse of energy to the repositioned vertically treated area.

18. The method of claim 17, wherein the step of reducing the tissue of the clitoral hood area further comprises completing additional passes of each first and second side with the heating device.

19. The method of claim 1, wherein the step of reducing the tissue of the periurethal area comprises placing the heating device superior of the periurethal area and applying a pulse of energy to a treated area.

20. The method of claim 19, wherein the step of reducing the tissue of the periurethal area further comprises repositioning the heating device to moderately overlap the treated area.

21. The method of claim 20, wherein the step of reducing the tissue of the periurethal area further comprises applying a pulse of energy to the repositioned treated area.

22. The method of claim 21, wherein the step of reducing the tissue of the periurethal area further comprises repeating the repositioning and heating steps until the entire area has been treated.

23. The method of claim 22, wherein the step of reducing the tissue of the periurethal area further comprises completing additional passes with the heating device.

24. The method of claim 1, wherein the step of reducing the tissue of the vaginal introitus area comprises placing the heating device directly on one side the vaginal area and applying a pulse of energy to a treated area.

25. The method of claim 24, wherein the step of reducing the tissue of the vaginal introitus area further comprises repositioning the heating device to moderately overlap the treated area.

26. The method of claim 25, wherein the step of reducing the tissue of the vaginal introitus area further comprises applying a pulse of energy to the repositioned treated area.

27. The method of claim 26, wherein the step of reducing the tissue of the vaginal introitus area further comprises repeating the repositioning and heating steps until the entire periphery of the vaginal opening area has been treated.

28. The method of claim 27, wherein the step of reducing the tissue of the vaginal introitus area further comprises completing additional passes with the heating device.

29. The method of claim 1, wherein the step of reducing the tissue of the perineal area comprises placing the heating device directly against skin of the perineum and applying a pulse of energy to a treated area.

30. The method of claim 29, wherein the step of reducing the tissue of the perineal area further comprises repositioning the heating device to moderately overlap the treated area.

31. The method of claim 30, wherein the step of reducing the tissue of the perineal area further comprises applying a pulse of energy to the repositioned treated area.

32. The method of claim 31, wherein the step of reducing the tissue of the perineal area further comprises repeating the repositioning and heating steps until the entire area has been treated.

33. The method of claim 32, wherein the step of reducing the tissue of the perineal area further comprises completing additional passes with the heating device.
